(19) United States
(12) Patent Application Publication
(10) Pub. No.: US 2009/0155875 A1
(43) Pub. Date: Jun. 18, 2009

(54) Methods to Enhance Carbon Monoxide Dehydrogenase Activity and Uses Thereof

(75) Inventors: David A. Berry, Brookline, MA (US); Robert S. Langer, Newton, MA (US); Richard J. Resnick, Somerville, MA (US)

Correspondence Address: WOLF GREENFIELD & SACKS, P.C., 600 ATLANTIC AVENUE, BOSTON, MA, 02210-2206, US

(73) Assignee: Massachusetts Institute of Technology, Cambridge, MA (US)

(21) Appl. No.: 11/884,522

(22) PCT Filed: Feb. 16, 2006
(86) PCT No.: PCT/US2006/005797

§ 371 Date: Oct. 24, 2008

Related U.S. Application Data
(60) Provisional application No. 60/653,728, filed on Feb. 16, 2005.

(51) Int. Cl. C12P 3/00; C12N 9/02; C12N 1/21

(52) U.S. Cl. 435/168; 435/189; 435/252.3

(57) ABSTRACT

This invention relates, in part, to methods and compositions for modulating the water-gas shift reaction (e.g., promoting the water-gas shift forward reaction) or in which the water-gas shift reaction has been modulated. The methods and compositions, therefore, also relate, in part, to increasing the oxidation rate of carbon monoxide (CO), for increasing the availability of CO (e.g., to the carbon monoxide dehydrogenase (CODH) enzyme complex), for removing and/or promoting the release of hydrogen and/or carbon dioxide (CO2), for regulating the redox potential of cells, for preventing free radical damage and/or promoting cell survivability, etc. The invention also relates, in part, to methods and compositions for modulating CODH activity, such as increasing CODH activity. Methods and compositions are also provided for modulating the PSII reaction (e.g., promoting the PSII forward reaction) or in which the PSII reaction has been modulated. The modulation of the PSII reaction can be in conjunction with the modulation of the water-gas shift reaction. The invention also relates, in part, to methods and compositions for modulating PSII activity, such as increasing PSII activity. The invention further relates to uses of the aforementioned methods and compositions. For example, methods and compositions are provided for the production of hydrogen and/or for the elimination of CO. The methods and compositions provided can be used for a variety of industrial and medical applications, and such applications are also provided as part of the invention.

### RELATED APPLICATIONS

This application claims priority under 35 U.S.C. §119 from U.S. provisional application Ser. No. 60/653,728, filed Feb. 16, 2005. The entire contents of which is herein incorporated by reference.

### GOVERNMENT SUPPORT

Aspects of this invention may have been made using funding from National Institutes of Health grant number 5-U19-CA052857-15. Accordingly, the government may have rights in the invention.

### FIELD OF THE INVENTION

This invention relates, in part, to the use of the water-gas shift reaction, such as that of photoheterotropic bacteria, in the production of hydrogen (hydrogen ions, H⁺, and/or dihydrogen, H₂) and/or the elimination of carbon monoxide (CO), where the production of hydrogen and/or the elimination of CO is enhanced by promoting the water-gas shift forward reaction. More specifically, this invention relates, in part, to methods and compositions for promoting the water-gas shift forward reaction, for example, by increasing the solubility of CO, preventing free radical damage and/or promoting cell survivability, regulating the redox potential of cells, removing and/or promoting the release of hydrogen and/or carbon dioxide (CO₂) and/or by providing oxygen (O₂). The invention also relates to methods and compositions for promoting the photosystem II (PSII) forward reaction. The invention further relates to uses of the aforementioned methods and compositions. For example, methods and compositions are provided for the production of hydrogen and/or for the elimination of CO. The methods and compositions provided can be used for a variety of industrial and medical applications, and such applications are also provided as part of the invention.

### BACKGROUND OF THE INVENTION

Hydrogen is currently produced by a variety of methods. Electrolysis and steam reforming of natural gas are most commonly used. Electrolysis uses electricity to induce the excitation of water (H₂O) to yield hydrogen and O₂ via the classic water-split reaction. Not only is this reaction expensive, but also, the power necessary to drive the reaction generally derives from the power grid and, therefore, depends on fossil fuels as its essential power source. Therefore, while the process of electrolysis is clean and sustainable, CO₂ is still released through the use of fossil fuels.

Steam reforming has a substantially lower cost profile than electrolysis and other available methods to produce hydrogen. The process of steam reforming utilizes natural gas or other light hydrocarbons that are catalytically converted into H₂ and CO. The CO is then converted into CO₂ endothermically, through the water-gas shift reaction. This CO₂ must be either sequestered or released. Sequestration increases cost, and release exacerbates the greenhouse gas effect.

Various photoheterotrophic bacteria, such as those within the Rhodospirillaceae family, can utilize CO as their carbon source to grow in the dark. In this process, CO can be oxidized to CO₂ using water to produce H₂ through a water-gas shift reaction (Formula 1) similar to the endothermic water-gas shift reaction used in steam reforming, although at ambient temperature. The water-gas shift reaction can be carried out by these organisms, or in the presence of catalysts. For instance, steam reforming methods employ “low temperature shift” catalysts to this reaction using compounds based on CuO/ZnO at temperatures between 180° C. and 280° C.

CO+H₂<—>CO₂+H₂ Formula 1

The oxidation of CO and production of H₂ through the water-gas shift reaction has a negative Gibbs free energy, consistent with a spontaneous forward reaction. The reaction is nonetheless catalyzed by an enzymatic pathway. In this pathway, CO is bound by carbon monoxide dehydrogenase (CODH) also known as carbon monoxide:acceptor oxidoreductase, which, a membrane bound protein itself, is part of a membrane bound complex consisting of eight putative gene products, including CODH. The ability to convert CO to H₂ using a bacterial system offers a way to increase H₂ production and minimize CO₂ output as well as an independent mechanism to use harmful gases to produce H₂.

CODH, present as part of an enzyme complex, performs the primary oxidation of CO to CO₂, directly yielding two hydrogen ions (H⁺) and two electrons (e⁻). The produced reducing equivalents are passed through a ferredoxin-like subunit, which is included within the bifunctional enzyme. The electrons are passed in an uncharacterized pathway to a tightly membrane-bound hydrogenase, where H₂ is formed. Bacterial oxidation of CO yielding H₂ can occur in darkness using CO as the sole carbon source, assimilating environmental CO from the environment into new cell mass. A limiting factor, however, is getting CO into a state where it can be used by the cell. Even in 100% CO atmosphere, vigorous shaking is required to enable cellular use.

### SUMMARY OF THE INVENTION

Methods and compositions are provided for promoting the water-gas shift forward reaction, for promoting the PSII forward reaction or some combination thereof. Methods and compositions are also provided for promoting CODH and/or PSII activity. The compositions provided include those in which or with which the above-mentioned reaction(s) are promoted and/or CODH and/or PSII activity is increased. Various applications for the methods and compositions provided are also included herein.

In one aspect of the invention a method for promoting the water-gas shift forward reaction is provided. Provided herein are a number of ways in which the water-gas shift forward reaction can be promoted. The method in some embodiments includes the step of increasing the solubility of CO in a sample, preventing free radical damage in a sample and/or promoting cell survivability, removing and/or promoting the release of hydrogen and/or CO₂ from a sample, adding O₂ to a sample or some combination thereof so that the water-gas shift forward reaction is promoted. In one embodiment the method results in enhanced hydrogen production. In another embodiment the methods results in enhanced CO elimination.

In another aspect of the invention a method is provided for promoting the water-gas shift forward reaction in a sample of cells. Again, provided herein are a number of ways to promote the water-gas shift forward reaction. The method in some embodiments includes the step of increasing the solubility of CO in a sample of cells, preventing free radical damage in a sample and/or promoting cell survivability, regulating the redox potential of cells in a sample, removing and/or promoting the release of hydrogen and/or CO₂ from the sample of cells, adding O₂ to the sample of cells or some combination thereof so that the water-gas shift forward reaction is promoted. In one embodiment the cells are CODH-containing cells. In another embodiment the cells are Rhodospirillum rubruin, Rhodopseudomonas gelatinosa or Carboxydothermus hydrogeiioformans cells. In another embodiment the method results in enhanced hydrogen production and/or CO elimination.

In still another aspect of the invention a method for promoting the water-gas shift forward reaction is provided. In one embodiment the water-gas shift forward reaction is promoted by providing an agent the promotes the water-gas shift forward reaction to a sample in an amount effective to promote the water-gas shift forward reaction. In another embodiment the sample is a sample containing CODH. In yet another embodiment the sample containing CODH is a sample of CODH-containing cells. In still another embodiment the cells are Rhodospirillum rubrum, Rhodopseudomonas gelatinosa or Carboxydothermus hydrogenoformans cells. In still another embodiment the agent that promotes the water-gas shift forward reaction is an agent that increases the solubility of CO, that prevents free radical damage and/or promotes cell survivability, that regulates the redox potential of cells, that removes and/or promotes the release of hydrogen and/or CO₂ or that adds O₂. In a further embodiment the water-gas shift forward reaction is promoted by providing some combination of the aforementioned agents.

In still a further embodiment the methods provided further comprise promoting the PSII forward reaction. In one embodiment, therefore, the methods further comprise providing an agent that promotes the PSII forward reaction. In another embodiment the PSII forward reaction is promoted by preventing the flow of electrons into the cytochrome b6f complex. In a further embodiment the PSII forward reaction is promoted by removing O₂. In still a further embodiment the PSII forward reaction is promoted by altering the redox potential of a cell in which the PSII reaction occurs. In yet another embodiment the PSII forward reaction is promoted by providing light (e.g., an optimal level of light). In one embodiment this is accomplished by the use of filters. In still another embodiment the PSII forward reaction is promoted by increasing the production of DI and/or other PSII components. In a further embodiment the PSII forward reaction is promoted by altering calcium levels.

In a further aspect of the invention a method for enhancing hydrogen production and/or CO elimination is provided. The method in one embodiment comprises promoting the water-gas shift forward reaction in a sample of CODH-containing cells so that hydrogen production and/or CO elimination is enhanced. In another embodiment the method further comprises promoting the PSII forward reaction.

In still another aspect of the invention a method of increasing the solubility of carbon monoxide (CO) in a sample containing CO is provided. In one embodiment the method comprises providing a CO-binding agent to a sample containing CO in an amount effective to increase the solubility of the CO. In another embodiment the method further comprises promoting the forward water-gas shift reaction. In one embodiment the water-gas shift forward reaction is promoted by removing and/or promoting the release of hydrogen and/or CO₂ from the sample or by adding O₂ to the sample. In another embodiment the sample containing CO is a culture of cells, and the water-gas shift forward reaction is promoted by regulating the redox potential of the cells. In still another embodiment the sample containing CO is a culture of cells, and the water-gas shift forward reaction is promoted by preventing free radical damage and/or promoting cell survivability.

In yet another aspect of the invention a method of increasing CODH activity is provided. In one embodiment the method comprises increasing the solubility of CO in a sample that contains CODH. In another embodiment the method comprises providing a CO-binding agent to a sample that contains CODH, wherein the CODH activity is increased. In one embodiment both CO solubility and CODH activity are increased. In still another embodiment the method of increasing CODH activity comprises removing and/or promoting, the release of hydrogen and/or CO₂ from a sample containing CODH, wherein CODH activity is increased. In yet another embodiment the method of increasing CODH activity, comprises providing O₂ to a sample containing CODH, wherein CODH activity is increased. In one embodiment of any of these methods CO release is also increased.

In a further embodiment the method of increasing CODH activity comprises providing to a sample containing CODH an agent the prevents free radical damage and/or promotes cell survivability, wherein CODH activity is increased. In one embodiment the agent that prevents free radical damage and/or promotes cell survivability is added directly to the sample. In another embodiment the agent that prevents free radical damage and/or promotes cell survivability is a vector for transfecting cells with a nucleic acid that codes for a protein that prevents free radical damage and/or promotes cell survivability. In still another embodiment the agent that prevents free radical damage and/or promotes cell survivability is provided to a sample by contacting the sample with one or more cells that produce, and preferably secrete, the agent.

In still another embodiment of the invention the method of increasing CODH activity comprises regulating the redox potential of CODH-containing cells in a sample, wherein CODH activity is increased. In one embodiment the CODH-containing cells are cultured with an agent that regulates the redox potential of the CODH-containing cells. In another embodiment the agent that regulates the redox potential of the CODH-containing cells is an agent that inhibits potassium ion entry, an agent that inhibits sodium ion efflux or is a reducing agent.

In yet another aspect of the invention a method for promoting the forward PSII reaction is provided. In one embodiment the method comprises promoting the forward PSII reaction in a sample. In another embodiment promoting the forward PSII reaction in a sample comprises providing an agent that promotes the forward PSII reaction to the sample. In another embodiment the PSII forward reaction is promoted by preventing the flow of electrons into the cytochrome b6f complex. In a further embodiment the PSII forward reaction is promoted by removing O₂. In still a further embodiment the PSII forward reaction is promoted by altering the redox potential of a cell in which the PSII reaction occurs. In yet another embodiment the PSII forward reaction is promoted by providing light (e.g., an optimal level of light). In one embodiment this is accomplished by the use of filters. In still another embodiment the PSII forward reaction is promoted by increasing the production of D1 and/or other PSII components. In a further embodiment the PSII forward reaction is promoted by altering calcium levels.

In one embodiment of any of the methods provided increasing the solubility of CO in a sample comprises providing an agent to the sample that increases the solubility of CO. In one embodiment the agent that increases the solubility of CO is a CO-binding agent. In another embodiment the CO-binding agent is an oxygen-carrying protein, a porphyrin-containing protein or a porphyrin. In a further embodiment the oxygen-carrying protein is a globin, such as hemoglobin or myoglobin. In one embodiment the agent that increases the solubility of CO is provided directly to the sample. In still another embodiment the agent is provided by contacting the sample with one or more cells that produce, and preferably secrete, the agent. IE a further embodiment the agent that increases the solubility of CO is a vector for transfecting one or more cells of the sample with a nucleic acid that encodes an agent that increases the solubility of CO. In one embodiment an oxygen-carrying protein or porphyrin-containing protein is provided by transfecting cells of a sample with a nucleic acid that codes for the oxygen-carrying protein or porphyrin-containing protein. In another embodiment the cells of a sample are CODH-containing cells (e.g., Rhodospirillum rubrum, Rhodopseudomonas gelatinosa or Carboxydothermus hydrogenoformans cells). In yet another embodiment an oxygen-carrying protein or porphyrin-containing protein is provided by transfecting the CODH-containing cells with a nucleic acid that codes for the oxygen-carrying protein or porphyrin-containing protein. In still another embodiment the CODH-containing cells are also transfected with a nucleic acid that codes for a protein that prevents free radical damage and/or promotes cell survivability.

In another embodiment preventing free radical damage and/or promoting cell survivability in a sample comprises providing to the sample an agent that prevents free radical damage and/or promotes cell survivability. In one embodiment the agent that prevents free radical damage and/or promotes cell survivability is catalase, superoxide dismutase, NADH-peroxidase or NADPH-peroxidase. In another embodiment the agent that prevents free radical damage and/or promotes cell survivability is a protein, and cells of a sample are transfected with a nucleic acid that codes for the protein. In still another embodiment preventing free radical damage and/or promoting cell survivability comprises providing to a sample one or more cells that produce, and preferably secrete, an agent that prevents free radical damage and/or promotes cell survivability.

In still another embodiment removing and/or promoting the release of hydrogen and/or CO₂ comprises providing to a sample an agent that removes and/or promotes the release of hydrogen and/or CO₂. In one embodiment the agent that removes and/or promotes the release of hydrogen and/or CO₂ is E. coli formate dehydrogenase, palladium or a bacterial or algal population that uses hydrogen and/or CO₂. In one embodiment the sample and the bacterial or algal population are intermixed. In yet another embodiment the sample and the bacterial or algal population are separated by a gas-permeable membrane. In a further embodiment the sample is a culture of CODH-containing cells, and the culture of CODH-containing cells is cultured with palladium. In another embodiment the sample is a culture of CODH-containing cells and the CODH-containing cells are cultured with E. coli formate dehydrogenase or one or more other cells that produce, and preferably secrete, E. coli formate dehydrogenase. In a further embodiment the culture of CODH-containing cells is cultured with a bacterial or algal population that uses hydrogen and/or CO₂. In one embodiment the culture of CODH-containing cells and the bacterial or algal population are intermixed. In another embodiment the culture of CODH-containing cells and the bacterial or algal population are separated by a gas-permeable membrane. In a further embodiment the CODH-containing cells are transfected with a nucleic acid that codes for E. coli formate dehydrogenase.

In yet a further embodiment adding O₂ to a sample comprises providing O₂ as pure gas to the sample. In another embodiment adding O₂ to a sample comprises contacting the sample with a bacterial or algal population that produces O₂ as a byproduct.

In another embodiment regulating the redox potential of one or more cells in a sample comprises providing an agent that regulates the redox potential of the cells. In one embodiment the agent that regulates the redox potential of one or more cells in a sample is an agent that inhibits potassium ion entry, an agent that inhibits sodium ion efflux or is a reducing agent.

Any of the methods provided herein can in one embodiment be or be part of a steam reforming process, bioremediation processes, liquid fuel production process, gaseous fuel production process or hydrogen fuel production process. In one embodiment the steam reforming process is a steam-methane reforming process. In another embodiment the bioremediation process is a dehalogenation, nitrate reduction or perchlorate reduction process. In still a further embodiment the liquid fuel production process is a process that includes coal conversion.

Any of the methods provided herein can in another embodiment be or be part of a CO elimination process. Any of the methods provided herein in one embodiment can result in or be used to result in enhanced hydrogen production and/or CO elimination. In one embodiment the enhanced hydrogen production is enhanced H₂ production.

Any of the methods provided can in another embodiment further include promoting the PSII forward reaction. In one embodiment the methods include the step of providing to a sample an agent that promotes the PSII forward reaction. Other methods for promoting the PSII forward reaction can also be used and are provided elsewhere herein.

In another aspect of the invention compositions are also provided in which or with which the water-gas shift forward reaction and/or the PSII forward reaction is promoted. In still another aspect of the invention compositions are provided in which or with which CODH and/or PSII activity is increased. In yet another aspect of the invention compositions are provided that comprise one or more agents that promote the water-gas shift forward reaction and/or one or more agents that promote the PSII forward reaction.

In a further aspect of the invention a composition comprising a sample containing CODH, wherein the water-gas shift forward reaction is promoted, is provided. In one embodiment the composition further comprises an agent that promotes the water-gas shift forward reaction. In another embodiment the CODH is the CODH of CODH-containing cells. In still another embodiment the PSII forward reaction is also promoted. In yet another embodiment the composition further comprises an agent that promotes the PSII forward reaction.

In another aspect of the invention a composition comprising a sample containing CO and an agent that increases the solubility of CO is provided. In another embodiment the composition further comprises an agent for promoting the water-gas shift forward reaction. In one embodiment the agent is an agent that prevents free radical damage and/or promotes cell survivability. In another aspect of the invention a composition comprising a sample containing CO and an agent that prevents free radical damage and/or promotes cell survivability is provided.

In still another aspect of the invention a CODH-containing cell transfected with a nucleic acid that codes for a CO-binding protein is provided. In one embodiment the CODH-containing cell is further transfected with a nucleic acid that codes for a protein that prevents free radical damage and/or promotes cell survivability. In another embodiment the CODH-containing cell is further transfected with a nucleic acid that codes for E. coli formate dehydrogenase.

In another aspect of the invention a CODH-containing cell transfected with a nucleic acid that codes for a protein that prevents free radical damage and/or promotes cell survivability is provided.

In still another aspect of the invention a CODH-containing cell transfected with a nucleic acid that codes for E. coli formate dehydrogenase is provided

In a further aspect of the invention a culture of any of the cells described herein is provided. In one embodiment the culture of cells can further comprise an agent that promotes the water-gas shift forward reaction. In another embodiment the culture of cells can comprise an agent that promotes the PSII forward reaction. In a further embodiment the culture of cells comprise both an agent that promotes the water-gas shift forward reaction and an agent that promotes the PSII forward reaction. In another embodiment the cells are CODH-containing cells.

In yet another embodiment the compositions provided can be used to enhance hydrogen production and/or CO elimination or are those in which enhanced hydrogen production and/or CO elimination results. In one embodiment the enhanced hydrogen production is H₂ production.

In a further embodiment the compositions provided include those in which or with which the water-gas shift forward reaction and/or PSII forward reaction is promoted. In another embodiment the compositions provided include those in which or with which CODH and/or PSII activity is increased. In another embodiment the compositions provided can include an agent that promotes the water-gas shift forward reaction or an agent that promotes the PSII forward reaction or both.

In a further aspect of the invention a method of enhanced hydrogen production and/or CO elimination is provided. In one embodiment the enhanced hydrogen production is enhanced H₂ production. In another embodiment the method comprises providing a CODH-containing composition in which the water-gas shift forward reaction is promoted to a composition in which enhanced hydrogen production and/or CO elimination is desired. In one embodiment the CODH-containing composition is one in which the PSII forward reaction is also promoted. In another embodiment the composition in which enhanced hydrogen production and/or CO elimination is desired is a composition in need of bioremediation. In still another embodiment the composition in which enhanced hydrogen production and/or CO elimination is desired is a composition that is part of a steam reforming process. In one embodiment the steam reforming process is a steam-methane reforming process. In a further embodiment the composition in which enhanced hydrogen production and/or CO elimination is desired is a composition that is part of a liquid fuel production process. In another embodiment the composition in which enhanced hydrogen production and/or CO elimination is desired is a composition that is part of a gaseous fuel production process. In yet another embodiment the composition in which enhanced hydrogen production and/or CO elimination is desired is a composition that is part of a hydrogen fuel production process. In a further embodiment the composition in which enhanced hydrogen production and/or CO elimination is desired is blood. In still an other embodiment the composition in which enhanced hydrogen production and/or CO elimination is desired is exhaust (e.g., vehicle exhaust).

In another embodiment of the invention the methods and compositions provided can include the addition of hydrogenase to promote the formation of H₂.

The samples of the methods and compositions provided can be in one embodiment samples containing CO, samples containing CODH or samples containing both CO and CODH. In another embodiment the sample contains cells. In a further embodiment the sample containing CO is a culture of cells. In still a further embodiment the sample contains cells and it is the cells that contain CODH. In one embodiment the sample is of CODH-containing cells. In another embodiment the sample containing CODH is a culture of CODH-containing cells. In another embodiment the cells are Rhodospirillum rubrum, Rhodopseudomonas gelatinosa or Carboxydothermus hydrogenoformans ells. In another embodiment the sample is a sample in which the water-gas shift reaction occurs, the PSII reaction occurs or both. In another embodiment the sample containing CODH is a sample of CODH-containing cells in which the water-gas shift reaction occurs and/or the PSII reaction occurs.

In a further embodiment of any of the methods provided an agent can be provided directly to a sample. In another embodiment the agent can be provided by contacting a sample with a cell that produces the agent. In a further embodiment the agent is a vector for transfecting one or more cells, wherein the vector comprises a nucleic acid encoding the agent.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 shows the market for hydrogen gas. Hydrogen can be used as a fuel, but this is a minimal amount of its current use. In addition to a being a possible fuel source, hydrogen is a commodity gas. Hydrogen gas currently has a market of over $18 billion per year. Oxygen is a byproduct with a $1 billion market.

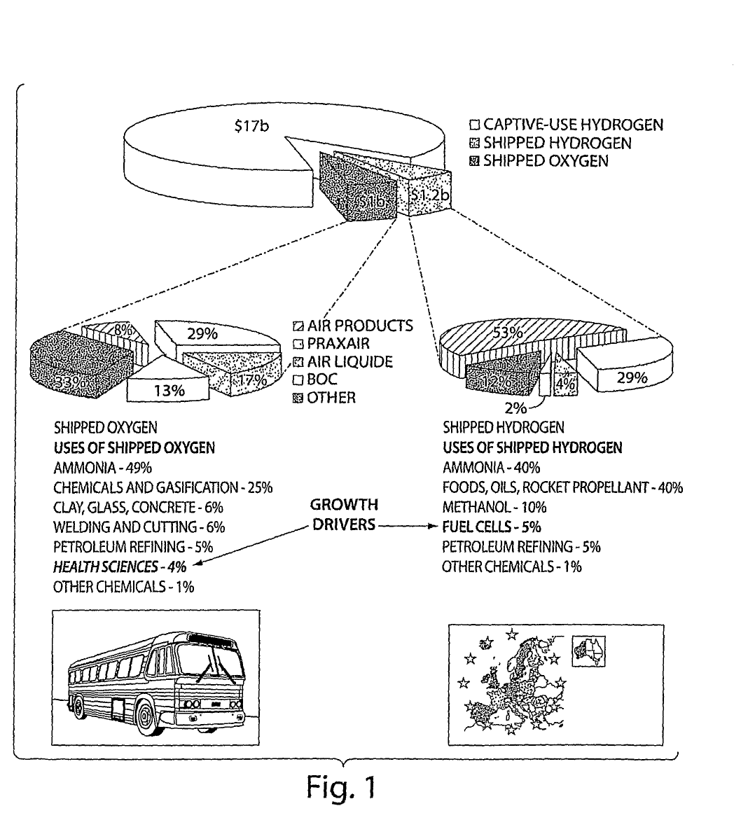

FIG. 2 shows various hydrogen production methods. Hydrogen is a commodity, and competition is based on price and purity. When carbon emissions are reduced in a process by carbon dioxide sequestration, the production costs are significantly increased, With a 10-fold increase in hydrogen production capacity, the costs could be competitive with steam reforming of natural gas (SMR), which is the market standard, but will not have fossil fuel dependence. In addition, improvements in hydrogen production can further reduce costs.

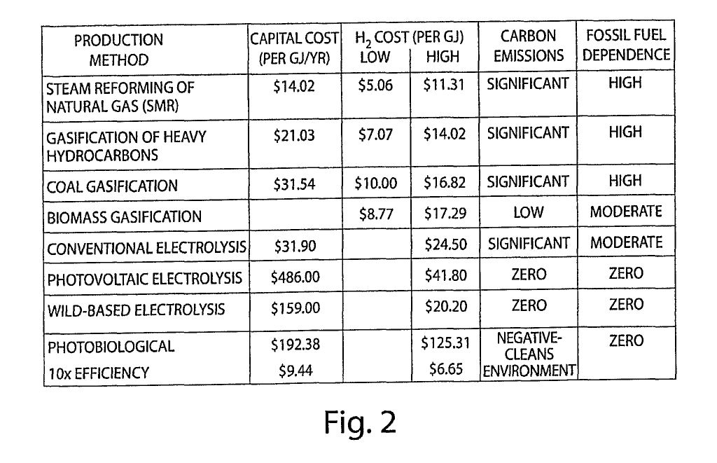

FIG. 3 shows the costs associated with various production methods.

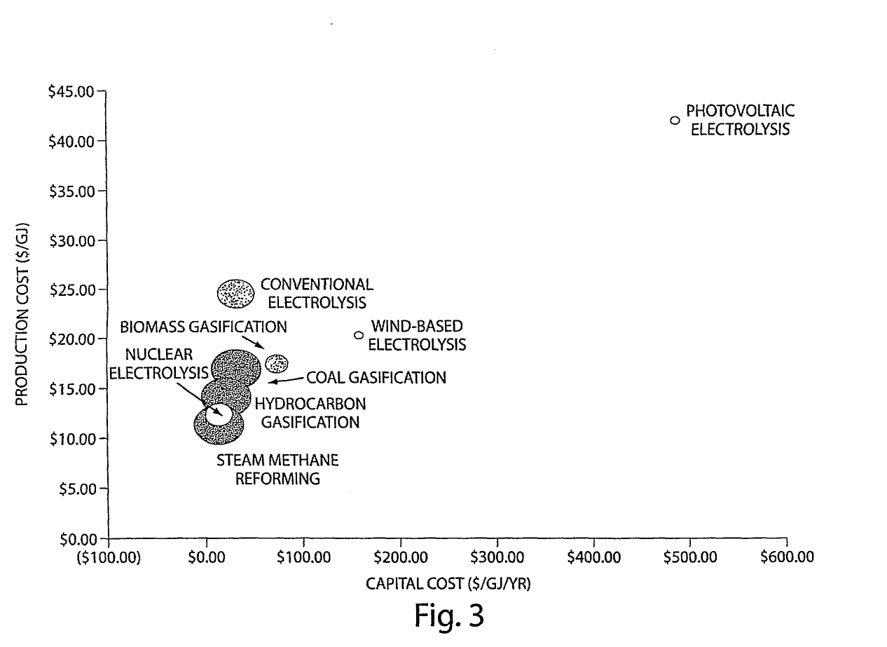

FIG. 4 illustrates the water-gas shift reaction. Currently, the water-gas shift reaction comprises 10-15% of the total operating cost of steam-methane reforming. The capital cost of water-gas shift is approximately $1.40/GJ/year, while the operating cost is approximately $1/GJ.

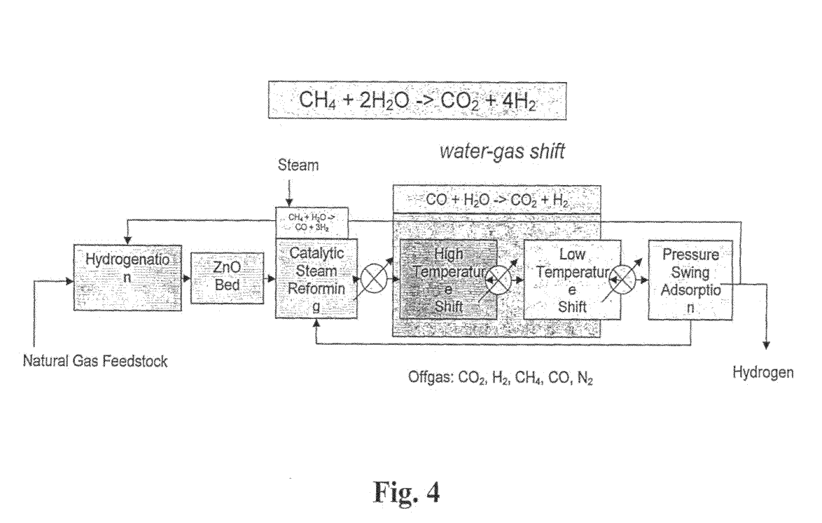

FIG. 5 shows the costs associated with water-gas shift. Using biological water-gas shift in steam-methane reforming reduces capital and production costs. A typical 50 SCFD plant could save $11 million per year. There are over 100 such plants in the United States.

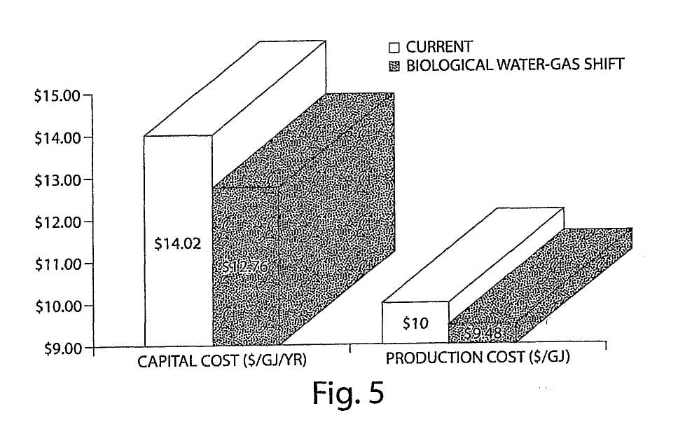

FIG. 6 shows a schematic of globin-mediated enhanced transfer of CO from the atmosphere to CODH. The biological water-gas shift reaction is catalyzed by CODH. Some bacteria with this enzyme can live exclusively off CO. Panel A provides a schematic of the normal reaction catalyzed by CODH and the associated protein complex. Panel B illustrates that the insertion of globins allows for binding of CO from the gaseous phase, which will increase the rate of oxidation and therefore H₂ production.

FIG. 7 illustrates photobiological hydrogen production. Water is normally split in nature in photosynthesis, which is an important process in plants and many bacteria. Bacteria contain processes to produce hydrogen gas from water split-products. The light reaction begins with photosystem II (PSII). Light causes charge separation, electrons are passed to produce NADPH, and water is split, yielding oxygen and hydrogen ions. An oxygen sensitive hydrogenase converts hydrogen ions into hydrogen gas. Increasing photosynthetic activity can increase the conversion of hydrogen ions to hydrogen gas.

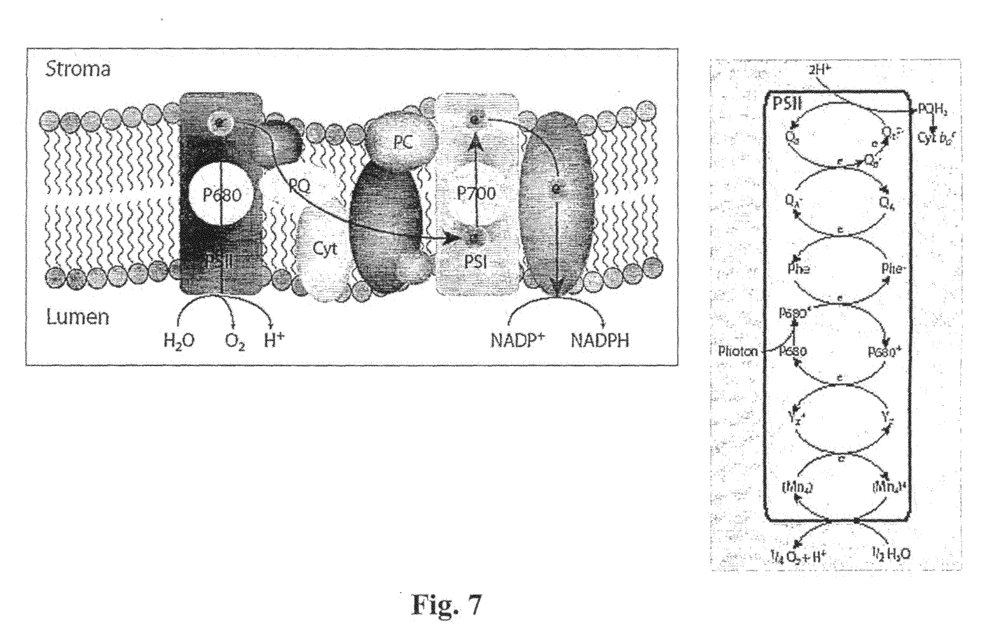

FIG. 8 provides the level of CODH and hydrogenase activity of R. rubrum, R. rubrum with soluble hemoglobin and R. rubrum expressing hemoglobin.

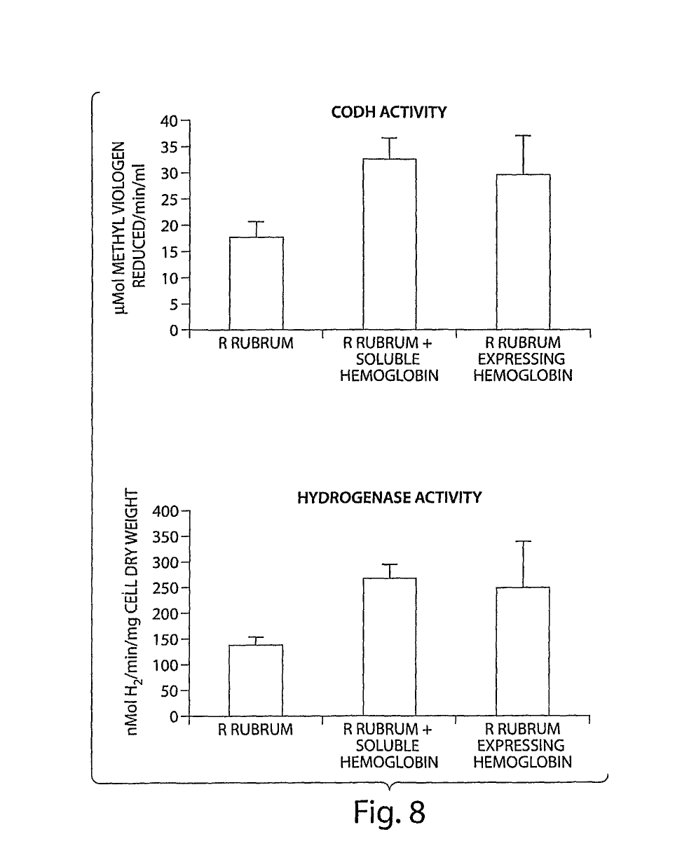

### DETAILED DESCRIPTION OF THE INVENTION

Methods and compositions are provided for promoting the water-gas shift forward reaction, for promoting the PSII forward reaction or some combination thereof. Methods and compositions are also provided for promoting CODH and/or PSII activity. The compositions provided include those in which or with which the above-mentioned reaction(s) are promoted and/or CODH and/or PSII activity is increased. Various applications for the methods and compositions provided are also included herein. This invention, therefore, provides methods and compositions for increasing hydrogen (H⁺ and/or H₂) production and/or CO elimination.

Oxidation of CO can occur by the water-gas shift reaction, such as the reaction that takes place in CODH-containing bacteria. The water-gas shift reaction is provided below as Formula 1. The mass transfer of CO can be enhanced in a number of ways, which include making CO more readily usable by CODH. The methods and compositions that have been discovered include, for example, methods and compositions that enable a cell to harness CO from the atmosphere, as well as methods and compositions that enhance the efficacy of the water-gas shift forward reaction. Therefore, provided herein are methods and compositions for promoting the water-gas shift forward reaction. The reaction is a reversible, oxidative reaction. To maximize CO oxidation and hydrogen output, the forward reaction must be maintained. “Promoting the water-gas shift forward reaction” is intended to refer to making the forward reaction, in which CO is oxidized, more favored. Promoting the water-gas shift forward reaction also refers to any increase in the rate of the forward water-gas shift reaction as well as any increase in the amount of the products of the forward reaction. Promoting the water-gas shift forward reaction also is intended to include promoting the primary oxidation of CO to Co₂, which yields two hydrogen ions (H⁺) and two electrons (e⁻), such as the reaction that is performed by CODH. The hydrogen ions can subsequently form H₂. Preferably, promotion of the water-gas shift forward reaction can result in hydrogen production (hydrogen ions (H⁺) and/or dihydrogen (H₂)) and/or CO elimination, and in some embodiments, hydrogen production and/or CO elimination is increased. As used herein an “agent that promotes the water-gas shift forward reaction” is any agent that can promote the water-gas shift forward reaction as defined herein. Such agents include agents that increase the solubility of CO, agents that prevent free radical damage and/or promote cell survivability, agents that regulated the redox potential of a cell, agents that remove and/or promote the release of hydrogen and/or CO₂, agents that add O₂, etc.

CO+H₂<—>CO₂+H₂ Formula 1

The water-gas shift forward reaction can be promoted by increasing the concentration of CO in the liquid phase. “Increasing the concentration of CO in the liquid phase” is intended to refer to any increase in the amount of CO that is present in the liquid phase. Such an increase can be accomplished by increasing the solubility of CO. “Increasing the solubility of CO” is intended to include any increase in the concentration of CO in the liquid phase and/or any increase in the amount of CO bound to soluble compounds, thereby increasing its likelihood of phase transfer to the liquid phase. Such increases can be accomplished with an agent that increases the solubility of CO. Such agents can be provided to a sample in which the water-gas shift reaction is occurring or can occur. As used herein, a “sample in which the water-gas shift reaction occurs” is intended to embrace a sample in which the reaction can occur but is not at that moment occurring as well as a sample in which the reaction is at that moment occurring. As used herein, “providing an agent to a sample” refers to contacting a sample with an agent. In some embodiments where the sample contains one or more cells the agent is placed in contact with the extracellular environment of the one or more cells. In another embodiment where the sample contains one or more cells the agent is introduced into the intracellular environment of one or more cells.

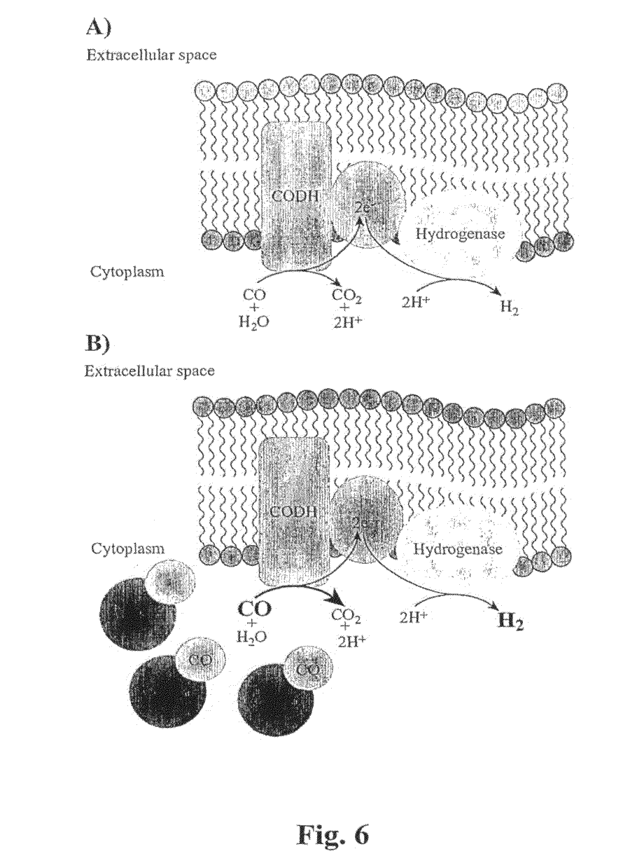

The solubility of CO can be increased with a CO-binding agent. In some embodiments the CO-binding agent is itself soluble. A “CO-binding agent” can be any molecule that can bind CO and that results in phase transfer or an increase in CO solubility. In some embodiments, it is preferred that the CO-binding agent binds CO with a higher efficiency than O₂. The CO-binding agents can serve as a biological sink for the CO. These agents can readily bind CO within the cell, at the membrane, or outside the cell, and can enable the mass transfer of CO from gas phase to soluble/liquid phase. These agents can, therefore, overcome a limiting factor in water-gas shift reactions, such as the CODH reaction. For example, the juxtaposition of CO-binding agent-bound CO to CODH can make more CO readily available to the enzyme, thus increasing the CO oxidized (FIG. 6) and hydrogen produced.

The methods provided herein, therefore, also include methods of increasing the solubility of CO in a sample containing CO. Such methods can include the step of providing a CO-binding agent to a sample in an amount effective to increase the solubility of CO. A “sample containing CO” is any sample in which CO is present. The CO in such samples can be in the gas or liquid phase or some combination thereof As used herein, “an amount effective to increase the solubility of CO” is any amount of an agent, such as a CO-binding agent, that alone or in combination with another agent or agents results in an increase in the solubility of CO in a sample relative to the solubility of CO in the sample prior to the addition of the agent or combination of agents.

CO-binding agents can include, for example, oxygen-carrying proteins. An “oxygen-carrying protein” is any protein that interacts with oxygen and can carry or transport it. Oxygen-carrying proteins include hemoglobin, myoglobin and other globins. Oxygen-carrying proteins, such as hemoglobin and myoglobin, can bind CO, often with higher efficiency than O₂. The terms “hemoglobin”, “myoglobin” and “globin” are intended to also refer to isoforms or modified versions of these proteins. For instance, hemoglobins include the hemoglobin isoforms (α, β, γ, δ and ε) and genetically modified versions of hemoglobin isoforms that promote multimer, e.g., tetramer, formation. Hemoglobins, therefore, also include engineered hemoglobins that include α-subunits with induced cross-linking of lysine residues within the central cavity to promote tetramer stability, β-subunits with N108K mutations to similarly promote tetramer stability, hemoglobin subunits bound to polyethylene glycol (PEG) to promote conjugation and stability, polymerized hemoglobin chains which pre-associate to form multiple hemoglobin subunits, etc. Hemoglobins, therefore, also include hemoglobins in which the proximal histidine has been mutated to increase CO binding to heme.

Methods for producing oxygen-carrying proteins, such as those described above, are known to those of ordinary skill in the art. For example, U.S. Pat. No. 5,840,851, by Plomer et al., describes methods to purify hemoglobin from cell lysates. U.S. Pat. No. 6,172,039, by DeAngelo et al., describes the expression of hemoglobin in non-mammalian cells. U.S. Pat. No. 5,798,227, by Hoffman et al., describes co-expression of multiple-hemoglobin subunits at the same time. U.S. Pat. No. 6,184,356, by Anderson et al., describes point mutations to stabilize globin multimers. U.S. Pat. No. 6,171,826, by Levine and Apostol describes methods to promote globin subunit dimerization. The teachings related to the production and expression of globins are incorporated herein by reference.

CO-binding agents also include other porphyrin-containing proteins as well as porphyrins themselves. A “porphyrin-containing protein”, as used herein, is any proteins that comprises a porphyrin. Porphyrin-containing proteins include, for example, hemoglobin, myoglobin, modified hemoglobins (e.g., sickle-cell hemoglobin (HbS) found in sickle cell anemia, hemoglobin H (HbH)/Hb Barts found in thalessemia), catalase, peroxidase, cytochrome c, chlorophyll a as well as other chlorophylls, etc. Porphyrins include, for example, coproporphyrin I dihydrochloride, chlorin e6, delta-aminolevulinic acid hydrochloride, bacteriochlorophyll a, bilirubin conjugate ditaurate disodium, bilirubin dimethyl ester, bilirubin (alpha), biliverdin dimethyl ester, biliverdin hydrochloride 3,6-bis(decyl)phthalonitrile, boron subphthalocyanine bromide, coproporphyrin III dihydrochloride, coproporphyrin I tetramethyl ester, coproporphyrin m tetramethyl ester, deuteroporphyrin IX dihydrochloride, deuteroporphyrin IX dimethyl ester, deuteroporphyrin IX 2,4-disulfonic acid dihydrochloride, deuteroporphyrin IX 2,4-disulfonic acid dimethyl ester disodium salt, deuteroporphyrin IX 2,4 bis ethylene glycol, Zn(II) deuteroporphyrin IX 2,4 bis ethylene glycol, deuteroporphyrin IX 2,4 (4,2) hydroxyethyl vinyl, deuteroporphyrin IX 2-vinyl 4-hydroxymethyl , 2,4-diacetyl deuteroporphyrin IX dimethyl ester, 2,4-diethyl deuteroporphyrin IX dimethyl ester, etioporphyrin I, hematoporphyrin IX base, hematoporphyrin IX dihydrochloride, hematoporphyrin IX dimethyl ester, hemin, heptacarboxylporphyrin I dihydrochloride, heptacarboxylporphyrin I heptamethyl ester, hexacarboxylporphyrin I dihydrochloride, hexacarboxylporphyrin I hexamethyl ester, isohematoporphyrin IX, mesobilirubin, Cr(III) mesoporphyrin IX chloride, Fe(III) mesoporphyrin IX chloride, Sn(IV) mesoporphyrin IX dichloride, mesoporphyrin IX dihydrochloride, mesoporphyrin IX dimethyl ester, N-methyl mesoporphyrin IX, N-methyl protoporphyrin IX, octaethylporphine, Ni(B) octaethylporphine, Pt(II) octaethylporphine (PtOEP), VO(IV) octaethylporphine, pentacarboxylporphyrin I dihydrochloride, pentacarboxylporphyrin I pentamethyl ester, pheophorbide a, phthalocyanine, phthalocyanine tetrasulfonic acid, Al(III) phthalocyanine chloride tetrasulfonic acid, Zn(II) phthalocyanine tetrasulfonic-acid, porphine, porphobilinogen, Co(III) protoporphyrin IX chloride, Mn(III) protoporphyrin IX chloride, Sn(IV) protoporphyrin IX dichloride, protoporphyrin IX dimethyl ester, protoporphyrin IX disodium salt , protoporphyrin IX, Zn(II) protoporphyrin IX, pyropheophorbide-a, pyropheophorbide-a methyl ester, stercobilin hydrochloride, tetrabenzoporphine, meso-tetra(4-carboxyphenyl)porphine, Cu(II) meso-tetra(4-carboxyphenyl)porphine, Fe(III) meso-tetra(4-carboxyphenyl)porphine chloride, Pd(II) meso-tetra(4-carboxyphenyl)porphine, meso-tetra(4-carboxyphenyl)porphine tetramethyl ester, meso-tetra(odichlorophenyl)porphine, meso-tetra[4-(dihydroxyboryl)phenyl]porphine, meso-tetra(heptafluoropropyl)porphine, meso-tetra(m-hydroxyphenyl)porphine, meso-tetra(p-hydroxyphenyl)porphine, meso-tetra(N-methyl-4-pyridyl)porphine tetra tosylate, meso-tetra(pentafluorophenyl)porphine chlorin free, Pt(II) meso-tetra(pentafluorophenyl)porphine, Pd(II) meso-tetra(pentafluorophenyl)porphin, meso-tetraphenylporphine, Ni(II) meso-tetraphenylporphine, VO(IV) meso-tetraphenylporphine, meso-tetra(4-pyridyl)porphine, meso-tetra(4-sulfonatophenyl)porphine dihydrochloride, Cu(II) meso-tetra(4-sulfonatophenyl) porphine, Fe(III) meso-tetra(4-sulfonatophenyl)porphine chloride, Mn(III) meso-tetra(4-sulfonatophenyl)porphine chloride, meso-tetra(4-N,N,N-trimethylanilinium)porphine tetrachloride, meso-tetra(2,4,6-trimethylphenyl)porphine, tetraazaporphine, urobilin hydrochloride, uroporphyrin I dihydrochloride, uroporphyrin III dihydrochloride, uroporphyrin I octamethyl ester and uroporphyrin III octamethyl ester.

The water-gas shift forward reaction can also be promoted by preventing free radical damage and/or promote cell survivability. “Free radical damage” includes any damage to one or more proteins in a sample, such as, for example, those of a cell in a sample, or to a cell itself as a result of one or more free radicals, such as hydrogen perodixide (H₂O₂), superoxide (O₂), etc. “Preventing free radical damage” refers to reducing or eliminating free radical damage. In some embodiments where the sample is one that contains one or more cells, the water-gas shift forward reaction is promoted by preventing free radical damage and/or promoting cell survivability. As used herein, to “promote cell survivability” means any improvement in the survival of a cell.

The agents that prevent free radical damage and/or promote cell survivability include proteins, such as catalase, superoxide dismutase, NADH-perodidase and NADPH-peroxidase. Damaging compounds are typically produced with the oxidation of heme, and high concentrations of hemoglobin can lead to cell death and reduce reproductive potential; therefore, the expression of these genes can increase the viability of, for example, proteins, such as oxygen-carrying proteins or porphyrin-containing proteins, and/or the lifespan of cells, such as the cells provided herein. The agents that prevent free radical damage and/or promote cell survivability can, in some embodiments, be introduced in combination with other agents, such as the CO-binding agents provided herein.

Any of the agents provided herein can be introduced alone or in combination with any of the other agents provided.

The water-gas shift forward reaction can also be promoted by removing and/or promoting the release of hydrogen and/or CO₂. Agents that remove and/or promote the release of hydrogen and/or CO₂ include, for example, palladium, E. coli formate dehydrogenase as well as bacterial and algal populations that use hydrogen and/or CO₂. These agents can be provided directly to a sample.

In some embodiments the bacterial or algal species use CO₂ as well as sunlight to promote photosynthetic activity. In other embodiments the other bacterial or algal species include Dechloromronas in the Beta Proteobacteria family, Dechloromnonas sp. strain HZ, strain KJ, strain PDX, strain JDS5, strain JDS6; Azospirillum in the Alpha Proteobacteria, Pseudomonas stutzeri DSM 50227, P. stutzeri (DSM 5190(I)), P. stutzeri strains DSM 50227 and DSM 5190(T), Dechlorormonas agitate and Escherichia coli 080. In some embodiments the other bacterial or algal species can be intermixed with a sample in which the water-gas shift forward reaction occurs. In other embodiments the other bacterial or algal species is separated by a gas-permeable membrane from a sample in which the water-gas shift forward reaction occurs.

E. coli formate dehydrogenase promotes the release of hydrogen and CO₂. In some embodiments, wherein the sample is a sample containing one or more cells, the addition of E. coli formate dehydrogenase is accomplished by the transfection of the cells with a plasmid or other vector expressing E. colt formate dehydrogenase. Where the water-gas shift reaction is the reaction performed by CODH, the release of hydrogen and CO₂ frees up the active sites on CODH, enabling future reactions to occur, and increasing the total reaction rate.

The water-gas shift forward reaction can also be promoted by facilitating the release of CO. CO release from proteins can be facilitated by bubbling O₂ through a sample. As an example, an environment in a sample can be created where no more than 80% of the gas present is CO. Oxygen can be supplied either as a pure gas or as a byproduct from another biological system, such as a bacterial or algal population. The bacterial or algal population can be intermized with a sample or separated from a sample by a gas-permeable membrane. Other ways to increase hydrogen or oxygen in a sample include cycling algae depth (facilitating liquid-air gas exchanging); agitating a system by stirring, shaking, rotating, etc.; removing carbon dioxide (in general, beyond just as associated with, for example, CODH); use of nitrogen fixing bacteria/algae; as well as vanadium, diphenylamine, hydrogen peroxide (in formic acid). These can be used in the methods and compositions provided herein.

In some embodiments the bacterial or algal population is one that includes cells that contain the photosystem II (PSII) complex engineered to have increased PSII activity. A “cell that has increased PSII activity” is one in which the PSII forward reaction is promoted, and there is an increased production of H₊, H₂ and/or O₂. The PSII reaction is provided in Formula 2. As used herein, “promoting the PSII forward reaction” is intended to mean making the PSII forward reaction more favored. Promoting the PSII forward reaction is intended to include increasing the rate of the forward reaction as well as increasing the amount of the products of the forward reaction that are produced. An “agent that promotes the PSII forward reaction” is intended to refer to any agent that promotes the PSII forward reaction as defined herein. Examples of such agents are provided below.

2H₂<13 >O₂+4H⁺ Formula 2

There are a number of ways in which PSII activity can be increased, and the equilibrium of the PSII reaction shifted such that the forward reaction is promoted and, in some embodiments, net H⁺ production is the result. The kinetics of an enzymatic process are regulated by the fundamental properties of the enzyme(s) as well as the concentrations of both substrates and products. By increasing the removal of products of the forward reaction, the equilibrium of the system will shift to speed up the process. Therefore, the removal of H⁺, either directly or indirectly, such as by conversion to H₂ (e.g., conversion of released H⁺ and E⁻ to H₂), can increase PSII activity and promote the forward reaction. As H⁺0 ions are generated, acidifying the cellular environment, electrons can be captured from various reactions inside or outside the cell and committed to the formation of H₂. This can be achieved through the structural modification of the PSII complex, via the addition of enzymes, or via some change to the exogenous cellular environment.

For example, hydrogenase can be added to a sample to increase PSII activity. Hydrogenase, a protein complex consisting of the structural proteins HoxH and HoxY, is known to be a bidirectional catalyst of the reaction 2H⁺+2e⁻<—>H2 (Formula 3). Through the addition of hydrogenase to the cellular environment, protons in the cytosol produced by PSII can be used to form H₂ readily. Hydrogenase, along with other enzymes mentioned herein, can be added directly to a sample or through genetic or structural modifications to a cell, such that the cell produces the enzyme.

In addition, membrane-degrading enzymes can be added to a sample to remove H⁺ from the sample and, therefore, promote the forward PSII reaction The production of H⁺ from the splitting of H₂O and the production of & are naturally separated by a phospholipid membrane. Controlled degradation of the membrane can allow for natural recombination of H⁺ and e⁻; promoting the removal of H⁺ the production of H₂. Membranes can be degraded using membrane-degrading enzymes, such as perforin, granzymes and lysozyme. “Membrane-degrading enzymes”, as used herein, are intended to refer to any enzyme that can degrade a cell membrane. Controlled production of these enzymes within cells can enable desirable membrane dysfunction without compromising cell viability. Furthermore, the production of these enzymes in a non-native species, including mouse or human, will prevent normal release into the extracellular space, allowing more efficient targeting of the desired membranes to promote H₂ production.

H⁺ can also be removed via the production of H₂ by preventing the flow of electrons into the cytochrome b6f complex, thereby rendering them available for immediate bonding with hydrogen ions produced during the PSII reaction.

The forward PSII reaction can also be promoted by the removal of O₂. For example, hydrolase can also be used to promote PSII activity. Hydrolases are proteins, such as salivary amylase and leukotrienes, that use water to cleave a substance. The use of hydrolases facilitates the photoconversion of sugars into H₂ and CO₂, providing a secondary source of H₂. More importantly for the purpose of promoting the forward PSII reaction, hydrolases can deplete the O₂ concentration within the cell, which can inactivate PSII function.

The PSII reaction equilibrium can additionally by regulated by changing the redox potential of a cell in which the PSII reaction occurs. Such alterations can lead to changes in photosensitivity of PSII, de-epoxidation state, and the capacity of linear electron flow. Manipulation of the redox potential of the system can be used to accelerate H⁺ production and reduce accumulation of e in cytochrome b6f. Furthermore, the direct transfer of energy due to light mediated activation of P680 occurs in picosecond time, while electron transfer via the quinone systems takes hundreds of milliseconds. Therefore, reducing dependence on the quinone system can reduce the effect of this rate-limiting aspect of the PSII reaction. For example, quinones and epoxides can be modified using enzymes such as epoxide hydrolase, NADPH reductase P450, DT diaphorase/quinone reductase to result in an increase in PSII activity. Introducing these enzymes to a sample, introducing cells that produce these enzymes to a sample or modifying PSII-containing cells genetically so that these enzymes are produced can serve as a functional block of the b6f complex. A “PSII-containing cell”, as used herein, is any cell in which the PSII complex is present and the PSII reaction occurs. A “cell in which the PSII reaction occurs” is any cell that contains a PSII reaction complex and in which the PSII reaction is occurring or can occur.

In addition, H⁺ and/or H₂ can be released from plastoquinone. Once electrons serve to reduce plastoquinone by bonding with 2H⁺ protons, these protons and/or the protons with associated electrons received from the double excitation of P680 can be released.

There are still other ways in which PSII activity can be increased. For example, filtering of sunlight strength can be used. PSII is initially activated by light energy, and the wavelength of the light is important in conferring activation. Therefore, UV-A and UV-B range wavelengths, but not UV-C wavelengths, can promote PSII activity. Light filters can be used in the methods provided to allow only optimal range light to a cellular environment. Furthermore, light pulses can be used for optimal saturation of PSII. Using filters to control the strength and duration of light entry into a sample environment can allow for maximal PSII activity by both supporting P680 activation and allowing for sufficient recovery time.

A PSII-containing cell can also be genetically modified to enable increased production of D1 and other PSII components. D1 is a protein normally found in PSII. Restoration of P680* to P680 can produce the formation of ³P₆₈₀ and triplet chlorophyll. These byproducts can interact with native oxygen to produce singlet oxygen, which can cause protein dysfunction. The short temporal lifespan of singlet oxygen makes D1 the most likely target of damage. Harming D1 and other proteins directly slows down the function of PSII, and secondarily, increases the rate of formation of triplet chlorophyll. Maintaining a higher quantity of functional proteins can maintain a higher level of functional PSII complexes per cell over time.

PSII activity can also be increased by altering calcium levels. Calcium (Ca²⁺) concentrations have been demonstrated to be important in regulating the reduction of P680* associated with electron transfer from Yz. Alterations in the levels of Ca²⁺ can, therefore, increase the rate of the PSII reaction, and increase the net production of H⁺ per unit time. Ca²⁺ levels can be regulated by cell surface ion transporters, by altering phosphate (PO₄³) levels to prevent salt production, and with the use of Ca²⁺ chelators such as ethylene glycol bis (beta-aminoethyl ether)-N,N,N′,N′-tetraacetic acid (EGTA) and ethylene diamine tetraacetic acid (EDTA).

PSII activity can also be increased with the addition of reducing agents. Reduction can be achieved using reducing agents that include 1,4-dithiothreitol (DTT), nicotine adenine dinucleotide (NAD), nicotine adenine dinucleotide phosphate (NADP), ascorbic acid, ferredoxin, methyl viologen and glutathione. These molecules can be added exogenously or produced by the expression of specific enzymes within the PSII-containing cell or another cell placed in contact with the PSII-containing cell. The addition of these compounds can alter cell redox potential, reduce transfer to the b6f complex and lower cellular oxygen concentrations, thereby promoting the PSII forward reaction.

Any of the methods and compositions provided herein can also include modifications to PSII, such as those described above. In some embodiments, the methods include promoting the water-gas shift forward reaction as well as the PSII forward reaction. In some of these embodiments the sample contains cells that contain CODH and PSII. In other of these embodiments the sample contains cells that contain CODH but not PSII as well as cells that contain PSII. In some embodiments the different cell populations are intermixed. In another embodiment the cell populations are separated by a gas-permeable membrane.

Where a sample contains one or more cells, such as those that contain CODH, the water-gas shift forward reaction can be promoted by regulating the redox potential of the one or more cells. The redox potential can be manipulated to create or maintain a negative potential by manipulating ion channels. The redox potential can also be manipulated by using redox agents, such as reducing agents, that promote the release of hydrogen. Examples of such reducing agents include ferrous ion, lithium aluminium hydride (LiAlH₄), potassium ferricyanide K₃Fe(CN)₆), sodium amalgam, sodium borohydride (NaBH₄), stannous ion, sulfite compounds, hydrazine, zinc-mercury amalgam (Zn(Hg)), diisobutylaluminum hydride (DIBAH), oxalic acid (C₂H₂O₄), etc. Ion channels can be manipulated to maintain a negative ion electric potential by inhibiting potassium ion (K⁺) entry with a potassium ion channel inhibitor. As −420 mV electric potential promotes release of H₂ from hydrogenase, the forward water-gas shift reaction can be promoted. Inhibitors of potassium ion entry include potassium channel inhibitors, such as, apamin precursor, antihypertensive protein BDS-I, antihypertensive protein BDS-II, charybdotoxin-like peptide Bs 6, contryphan-Vn, kappa-conotoxin BtX precursor, kappa-A-conotoxin SIVA precursor, kappa-conotoxin PVIIA precursor, venom basic protease inhibitor B, venom basic protease inhibitor I, venom basic protease inhibitor K, etc. Similarly, the ion channels can be manipulated to maintain a negative ion electric potential by inhibiting sodium ion (Na⁺) efflux with a sodium channel inhibitor. Sodium channel inhibitors include, for example, neurotoxin ANC45C1, neurotoxin AEP precursor, neurotoxin AEP2 precursor toxin AETX I, toxin APETx2, babycurus-toxin 1, birtoxin, delta-conotoxin Am 2766, delta-conotoxin GmVIA, delta-conotoxin PVIA precursor (Lockjaw peptide), delta-conotoxin NgVIA, delta-conotoxin EVIA, delta-conotoxin TxVIA precursor, delta-conotoxin TxVIB, conotoxin CcTx, conotoxin GS, etc.

Methods of increasing the solubility of CO in a sample are also provided. Such methods can include the step of providing a CO-binding agent to a sample in an amount effective to increase the solubility of CO. In some embodiments where the sample is one that contains CODH, the activity of the CODH is increased by providing a CO-binding agent to a sample. In some embodiments hydrogen (i.e., H⁺ and/or H₂) is produced. The CO-binding agent can be provided to the sample directly. The CO-binding agent can also be provided to the sample through the introduction to the sample of one or more cells that produce, and preferably secrete, the agent. The agent can also be a vector that comprises a nucleic acid molecule that codes for a CO-binding protein. The vector can be placed in contact with a sample so that one or more cells in the sample are transfected with the vector.

It has also been discovered that the activity of CODH can be modulated to, preferably, lead to the production of hydrogen and/or the elimination of CO. Therefore, this invention relates; in part, to methods and compositions for modulating, and in particular increasing, the activity of CODH. As used herein, “modulating CODH activity” refers to any change in the activity of the enzyme either directly or indirectly. The term is meant to include any change to the enzyme itself. The term, however, is also meant to include any change that affects the water-gas shift reaction, such as increasing the oxidation rate of carbon monoxide (CO) to carbon dioxide (CO₂) with the concomitant production of hydrogen ions (H+) and, in some embodiments, dihydrogen (H₂) through the action of the enzyme complex carbon monoxide dehydrogenase (CODH). Therefore, the term includes changes that affect the presence/availability of the reactants, products and/or rate of the water-gas shift reaction of CODH. The term also includes changes to the redox potential of cells containing CODH and in which the water-gas shift reaction occurs. The methods and compositions provided, for example, can increase the activity of CODH, which can result in the enhanced elimination of CO and/or the enhanced production of hydrogen.

Modulating the activity of CODH, therefore, includes making CO more readily available to the enzyme. The activity of CODH is primarily limited by the availability of CO, defined, at least in part, by the capacity to enable mass transfer between gas and liquid. CODH activity, in one aspect of the invention, can be modulated through the increase of the solubility of CO, such as by the introduction of CO-binding agents. The CO-binding agents can be provided to a sample containing CODH. A “sample containing CODH” is any sample that CODH can be found therein and, preferably, in which the water-gas shift reaction occurs. In one embodiment the CODH-containing sample is a sample of cells containing CODH. The providing of an agent to a sample can be merely placing the sample in contact with the agent. In some embodiments an agent is placed in contact with the extracellular environment of one or more cells of a sample. In another embodiment the agent is introduced into the intracellular environment. In still other embodiments the agent is produced by a cell, and the cell is placed in contact with a sample.

Also included herein, therefore, are methods for modulating CODH activity. In particular, the methods and compositions relate to increasing the activity of CODH. These methods can include the step of providing to a sample any of the agents provided herein that promote the water-gas shift forward reaction or some combination thereof. These methods can further include the agents described herein that promote the PSII forward reaction. Preferably, in the methods provided H⁺ and/or H₂ is produced, an-increased amount of H⁺ and/or H₂ is produced and/or CO is eliminated. The methods and compositions can also relate to enhancing the availability of carbon monoxide (CO) to CODH in order to promote the oxidation of CO and concomitant production of hydrogen ions (H⁺) and/or dihydrogen (H₂). The methods and compositions provided herein can also include increasing the oxidation rate of carbon monoxide (CO), increasing the availability of CO (e.g., to the CODH enzyme complex), removing and/or promoting the release of hydrogen and/or CO₂, adding O₂, and regulating the redox potential of a cell. The methods provided can, in some embodiments, include any combination of the foregoing.

The samples, as provided herein, includes samples containing one or more cells. The samples, as provided herein, also includes samples containing CO, CODH or both. In some embodiments the samples contain one or more cells, and it is the cells that contain CODH. Cells containing CODH can be any cell that contains CODH. For example CODH-containing cells include those of the Rhodospirillaceae family. CODH-containing cells include, for example, Rhodospirillum rubrum, Carboxydothermus hydrogenoformans, Rhodopseudomonas gelatinosa, Rhodocyclus gelatinosus and Clostridium thermoaceticum. These and other cells as provided herein can be transfected, for example, with plasmids or other vectors containing the genes for any of the proteins described herein, such as human or murine hemoglobin monomers or dimers, engineered hemoglobin monomers to promote the formation of dimers or tetramers, myoglobin, etc.

The bacterial species Rhodospirillum rubrum uses a CODH that specifically catalyzes the oxidation of CO without participating in a second functionality. These bacteria can survive on CO alone, and have a CO-activated hydrogenase. Carboxdothermus hydrogenoformans also express a similar CODH. The role of CODH, however, is not limited to the water-gas shift reaction, and the specific reactions catalyzed by CODH are species-specific. CODH from Moorella thenioacetica is bifunctional, containing a second site that combines CO, coenzyme A (CoA) and a methylated corrin-iron-sulfur protein to either synthesize acetyl-CoA or catabolize acetate. CODH from both Rhodospirillum rubrum and Carboxydothermus hydrogenoformans are monofunctional, catalyzing only the water-gas shift reaction. The CODH of Rhodospirillum rubrum, however, can also enable anaerobic growth, with the use of CO as a source of energy. In Rhodospirillum rubrum, the activity of the hydrogenase is promoted by CO, as is the production of CODH. The fact that this hydrogenase is not inhibited by CO is not common with other-hydrogenases. Interestingly, both the CO oxidation and the release of H₂ occur on the cytoplasmic side of the cell membrane.

The agents provided above and elsewhere herein can be introduced directly to a sample. When the sample is a sample containing one or more cells, the agents can be introduced to the extracellular or intracellular environment of the one or more cells. When the agent is a protein and the sample is one that contains one or-more cells, the agent can be a vector for transfecting the one or more cells, wherein the vector comprises a nucleic acid encoding the protein. Cells containing CODH, in one embodiment, are transfected with such a vector. The CO-binding agents, such as the oxygen-carrying proteins or porphyrin-containing proteins, for example, can be introduced by transfection. As another example, cells containing CODH can be transfected with genes (e.g., bacterial genes) to produce catalase, superoxide dismutase, NADH-perodidase and NADPH-peroxidase. As mentioned elsewhere herein, these proteins can prevent damage from hydrogen perodixide (H₂O₂), superoxide (O₂⁻) and other free radicals. For example, catalase or superoxide dismutase are expressed in red blood cells to prevent damage to the proteins from hydrogen perodixide (H₂O₂) and superoxide (O₂⁻), respectively. In one embodiment the cells can be transfected to produce a protein that prevents free radical damage and/or promotes cell survivability, such as catalase or superoxide dismutase, as well as a CO-binding protein.

In another embodiment another cell is transfected and placed in contact with a sample. In still another embodiment the cell and/or the nucleic acid is engineered such that the protein agent is secreted. In still another embodiment a cell is transfected with a nucleic acid encoding the protein agent, and a fraction of the cell is collected containing the protein agent subsequently produced. It is the fraction of the cell in this embodiment that is placed in contact with the sample. In yet another embodiment the nucleic acid is engineered so that the protein is excreted, and the protein is collected and placed in contact with the sample.

Cells can be transfected with plasmids, such as plasmids containing the genes for any of the protein agents provided herein, which include, for example, human or murine hemoglobin monomers or dimers, engineered hemoglobin monomers to promote formation of dimers or tetramers, or myoglobin under constitutive promoters or end-product inhibited promoters that enable the production of the proteins. The genes can be modified to promote gene excretion or membrane localization. Vectors can be constructed such that the proteins can be free in the cytoplasm (e.g., without additional genetic alterations), membrane associated (erg., adding an N-terminus hydrophilic domain) or secreted (e.g., adding the leader sequence from fibroblast growth factor 1 to the N-terminus). The cells that are transfected can be any cell for use in the methods and compositions provided. Such cells include, for example, Rhodospirillum rubrum, Rhodopseudomonas gelatinosa and Carboxydothermus hydrogenoformans cells.

As an example, the-insertion of the genes that encode CO-binding agents, when such agents are proteins, into cells can serve to bind CO and serve as a biological sink. The CO-binding proteins can readily bind CO within the cell, at the membrane, or outside the cell, and can enable the mass transfer of CO from gas to soluble. The juxtaposition of globin-bound CO to CODH can enable it to be more readily available to the enzyme. This modification can, therefore, facilitate the transfer of CO to a state usable by CODH. For example, the increased presence of CO binding sites through the expression of globin proteins can increase the transfer of CO from the gaseous state to a state where they are usable by CODH. By increasing the presence of substrate (CO), the forward reaction, the oxidation of CO with the concomitant production of hydrogen, can be promoted.

The plasmids and other vectors provided herein can contain constitutive promoters (e.g., cytomegalovirus (CMV) promoter, cassava vein mosaic virus (CsVMV) promoter, the sugarcane bacilliform badnavirus (ScBV) promoter, the 35S promoter, figwort mosaic virus (FMV) promoter, CsVMV promoter, alfalfa small subunit rubisco (RbcS) promoter, H3 or β actin promoter, arabinose-inducible araBAD promoter (PBAD) of Escherichia coli, galP2) or end-product inhibited promoters (e.g., trp and lac operon promoters) to enable the production of proteins. In addition, the genes can be additionally modified to promote gene excretion or membrane localization.

It will also be understood that the invention embraces the use of the sequences in expression vectors, as well as to transfect other host cells and cell lines, be these prokaryotic (e.g., E. coli), or eukaryotic (e.g., dendritic cells, chinese hamster ovary (CHO) cells, COS cells, yeast expression systems and recombinant baculovirus expression in insect cells). The expression vectors require that the pertinent sequence, i.e., those described supra, be operably linked to a promoter.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but- are not limited to, plasmids, phagemids and virus genomes. A cloning vector is one which is able to replicate autonomously or after integration into the genome in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a-promoter sequence for transcriptional control of the operably joined gene. Regulatory sequences may also include enhancer sequences or upstream activator sequences as desired. The vectors of the invention may optionally-include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989.

The invention also embraces so-called expression-kits, which allow the artisan to prepare a desired expression vector or vectors. Such expression kits include at least separate portions of at least two of the previously discussed materials. Other components may be added, as desired.

Also provided herein are compositions comprising any combination of any of the samples and agents provided herein. For example, compositions in which the water-gas shift forward reaction is promoted are provided. Compositions in which the PSII forward reaction is promoted are also provided. In one embodiment the compositions are compositions in which both the water-gas shift forward reaction and the PSII forward reaction are promoted. The compositions, therefore, include compositions that comprise an agent that promotes the water-gas shift forward reaction, an agent that promotes the PSII forward reaction, an agent that increases CODH activity, an agent that increases PSII activity or some combination thereof. In another embodiment the compositions can be used to produce hydrogen and/or eliminate CO. In still other embodiments hydrogen production and/or CO elimination occurs or is enhanced in the compositions. In some embodiments the compositions can contain CODH. In other embodiments the compositions provided include cells, such as cells containing CODH.

Also provided herein is a cell that is engineered to contain an agent that promotes the water-gas shift forward reaction, an agent that promotes the PSII forward reaction, an agent that increases CODH activity, an agent that increases PSII activity or some combination thereof. Preferably, the cell produces the agent or agents. In one embodiment the cell is a CODH-containing cell. In another embodiment the cell is transfected with a nucleic acid that encodes a CO-binding protein and/or a protein that prevents free radical damage and/or promotes cell survivability. Cultures of the cells described herein are also provided.

The methods and compositions described herein can be used in cell culture systems. The cells of the cell culture systems can be, for example, any cell, such as a cell that expresses CODH.(i.e., naturally expresses or is engineered to express CODH) and/or PSII. The cells include the bacterial species of the Rhodospirillaceae family. The bacterial species of the Rhodospirillaceae family include, but are not limited to, Rhodospirillum rubrum, Carboxydothermus hydrogenoformans, Rhodopseudomonas gelatinosa and Moorella thermoacetica. CODH-containing cells include, for example, Rhodospirillum rubrum, Carboxydothermus hydrogenoformans, Rhodopseudomonas gelatinosa, Rhodocyclus gelatinosus, and Clostridium thermoaceticum.

Also provided herein are the cells described as well as cultures containing the cells. The cell cultures can, optionally, contain any of the agents provided herein for promoting the water-gas shift forward reaction, for increasing CODH activity, for promoting the PSII forward reaction, for increasing PSII activity or some combination thereof.

Cells can be cultured in an appropriate nutrient medium under conditions that are metabolically favorable for the growth of the cells. As used herein, the phrase “metabolically favorable conditions” refers to conditions that maintain cell viability. Such conditions include growth in nutrient medium at 37° C. in a 5% CO₂ incubator with greater than 90% humidity. Many commercially available media, such as RPMI 1640, Fisher's, Iscove's, McCoy's, Dulbecco's Modified Eagle's Medium, etc., and the like, which may or may not be supplemented with serum, may be suitable for use as nutrient medium. In general, cell suspensions can be cultured according to standard cell culture techniques. On a small scale, the cultures can be contained in culture plates, flasks and dishes. On a large scale, the cultures can be contained in roller bottles, spinner flasks and other culture vessels, such as fermenters. Culturing in a three-dimensional, porous, solid matrix can also be used.

The methods and compositions provided herein can result in or be used in the production of H⁺ and/or H₂. The methods and compositions provided can also result in or be used in CO elimination. Methods and compositions are provided for these results. As used herein, the “enhanced elimination of CO” is meant to refer to an increase in the elimination of CO using the methods and compositions provided herein as compared to when the methods and compositions provided are not used. As used herein, “enhanced production of hydrogen” refers to an increase in the production of hydrogen ions and/or dihydrogen using the methods and compositions provided as compared to when-the methods and compositions provided are not used. Both the elimination of CO and production of hydrogen have substantial utility, both medically and industrially.

The invention, therefore, also provides industrial and medical applications using the methods and compositions provided herein. These applications include, for example, removing CO from the blood, treating CO poisoning, bioremediation, decreasing CO emissions and producing-hydrogen gas. Hydrogen gas that is produced can be used in, for example, steam reforming processes, such as steam-methane reforming, liquid fuel production processes, gas fuel production processes and hydrogen fuel production processes. Such applications using the methods and compositions described herein are also provided. Therefore, the methods and compositions provided herein are or can be used as part of a steam reforming process, a bioremediation processes, a liquid fuel production process, a gaseous fuel production process, a hydrogen fuel production process or a CO elimination process.

Therefore, also provided herein is a method of enhanced hydrogen production or CO elimination, wherein any of the compositions provided are provided to a composition in which enhanced hydrogen production or CO elimination is desired. A “composition in which enhanced hydrogen production or CO elimination is desired” is any composition in which the production of hydrogen ions and/or dihydrogen and/or the elimination of CO is beneficial. Such a composition includes a composition in need of bioremediation, a composition that is part of a steam reforming process (e.g., a steam-methane reforming process), a composition that is part of a liquid fuel production process, a composition that is part of a gaseous fuel production process, a composition that is part of a hydrogen fuel production process or a composition that is in need of CO reduction or elimination (e.g., blood or exhaust, such as vehicle exhaust). In one embodiment the method comprises providing a CODH-containing composition in which the water-gas shift forward reaction is promoted to the composition in which enhanced hydrogen production or CO elimination is desired. In another embodiment the CODH-containing composition is one in which the PSII forward reaction is also promoted.

The methods and compositions provided, therefore, can be used in the treatment of CO poisoning. Medically, CO poisoning occurs because hemoglobin has a higher affinity to CO than it does to O₂. Treatment can serve to remove the CO and allow oxygen to continue to return to the hemoglobin binding sites. The methods and compositions provided can be used to remove CO from a sample. The methods and compositions provided can also be used as part of a bioreactor wherein blood, including the hemoglobin-containing red blood cells, would be brought into the bioreactor, CO would be effectively removed from the hemoglobin, and the blood would be able to be returned to the body of a subject. Therefore, methods are provided herein that comprise contacting blood containing CO with a composition as provided herein to result in the elimination of CO.

The compositions provided can also be used to reduce CO emissions from vehicles and CO-producing factories, e.g., with the culture systems provided herein surrounding exhausts. Samples containing CO, such as exhaust, can be contacted with the compositions provided herein, such that the CO levels are reduced or eliminated from the sample. Such a method is also provided.

Methods and products are also provided wherein hydrogen gas is produced. Steam reforming is a technique currently used to produce industrial hydrogen. Current methods employ “low temperature shift” catalysts to this reaction using compounds based on CuO/ZnO at temperatures between 180° C. and 280° C., in a petroleum-dependent manner. Given recent worldwide environmental problems and dependence on other countries, the U.S. government has prompted federally supported programs and projects to produce hydrogen in an inexpensive, sustainable manner. The methods provided offer ways to enhance the efficiency of hydrogen gas production while reducing the associated pollution of steam reforming. Also provided are methods to produce hydrogen independent of fossil fuels that exhibits long-term sustainability. For example, bacteria in which the water-gas shift forward reaction and/or PSII forward reaction is promoted, such as bacteria engineered to have enhanced CODH and/or PSII activity, can be used in several settings to produce hydrogen. For example, such bacteria can be used in steam reforming to perform the water-gas shift reaction. This reaction is normally catalyzed by cationic metals and operates under high temperatures. The use of the methods and compositions provided herein, such as the engineered cells or compositions containing cells, will not require additional supplements beyond that used in steam reforming, can operate at ambient temperature, and can enable the same or increased output without the need for exogenous metals. The methods additionally have advantages over electrolysis, the current alternative to steam-reforming, as electricity from the power grid can be greatly reduced. Furthermore, the methods have higher fundamental efficiency than photobiological production of hydrogen, which has been thought of as a distant but potential replacement for steam reforming and electrolysis.

The engineered cells or the compositions provided containing cells can be used for enhanced H⁺ and/or H₂ production. This can serve directly as a source for H₂ to-be isolated. The increased hydrogen production can also be used in a co-culture setting such that the hydrogen can directly provide necessary nutrients to other species to promote bioremediation processes including dehalogenation, nitrate reduction and perchlorate reduction. Therefore, bacterial or algal species that consume hydrogen, such as H₂, can be co-cultured with the cells and compositions provided herein. The cell populations can be intermixed or separated by a gas-permeable membrane. Such bacterial or algal species include Dechloromonas in the Beta Proteobacteria family, Dechloromonas sp. strain HZ, strain KJ, strain PDX strain JDS5, strain JDS6; Azospirillum in the Alpha Proteobacteria, Pseudomonas stutzeri DSM 50227, P. stutzeri (DSM 5190(T)), P. stutzeri strains DSM 50227 and DSM 5190(T), Dechloromonas agitate and Escherichia coli 080.

A subject is any human or non-human vertebrate, e.g., dog, cat, horse, cow, pig.

The invention will be more fully understood by reference to the following examples. These examples, however, are merely intended to illustrate the embodiments of the invention and are not to be construed to limit the scope of the invention.

### EXAMPLES

### Materials and Methods

### Globin Production

The human α- and β-globin chains were encoded in a pHE2 plasmid as described (Shen, T. J., et al. (1993) Proc Natl Acad Sci USA 90: 8108-12). This plasmid was transfected into algal species as appropriate for expression assays or into E. coli to produce exogenous protein. In all cases, DNA sequences were verified, and the produced protein was confirmed to be that desired by mass spectrometry as described (Shen, T. J., et al. (1993) Proc Natl Acad Sci USA 90: 8108-12) as well as by Western blot for each the αa- and β-globin chains. Protein expression was induced in both E. coli and algal species by the addition of 0.2 mM ispropyl β-thiogalactopyranoside (IPTG). Cells were supplemented with 20 mg/L hemin and 10 g/L glucose during the expression period. Cells were allowed to grow for four hours prior to experimentation or purification as appropriate.

In E. coli specifically, soluble hemoglobin was expressed and purified as described (Bobfchak, K. M., et al. (2003) Am J Physiol Heart Circ Physiol 285: H549-61) with slight modification. Cells were grown in glucose-supplemented DM-4 medium in a 10 liter fermenter (Looker, D., et al. (1994) Methods Enzymol 231: 364-74). Protein expression was performed as described. Subsequently, the cells were equilibrated with CO, harvested, and lysed by treatment with 1 mg/ml lysozyme, 50 μl/l bezonase followed by gentle sonication on ice. The resulting slurry was centrifuged at 4° C. for 45 min at 14,000 rpm. The supernatant was saturated with CO, adjusted to pH 8.0 with 1M Tris base and treated with 10% polyethyleneimine to yield a final concentration of 0.5% (vol/vol) in order to precipitate out nucleic acids. Hemoglobin Was then purified by a two-step ammonium sulfate precipitation (45% and 75%), followed by chromatography using a CM SepharoseFF column (Amersham Pharmacia Biotech, Piscataway, N.J.). The protein and the column were equilibrated with 1 mM benzamidine, 0.3 mM dithiothreitol, 1 mM EDTA, 0.1 mM triethylenetetramine in 10 mM phosphate buffer, pH 6.5. Purity of the hemoglobin was confirmed by reversed-phase HPLC which identified the presence of heme by a peak with an elusion time of 11.6 minutes. A peak at 46.9 minutes corresponded to α-globin and one at 73.4 minutes was β-globin. UV spectra further confirmed the absence of other proteins or contaminating nucleotides or sugars (232 nm).

Spontaneous polymerization through the oxidation of thiol groups leading to S—S bonds between Cys residues at the β9 position was carried out in 20 mM Tris, 1.0 mM benzamidine, 0.1 mM triethylenetetramine, pH 8.4, under 1 ATM O₂. Polymerization was confirmed, and the extent determined with a prepacked (1.6×60 cm) Fractogel EMD BioSec column (EM Industries, Hawthorne, N.Y.) using a 20 mM phosphate, 300 mM NaCl, pH 7.2 elution buffer. Methemoglobin was reduced as described (Hayshi, A., et al. (1973) Biochim Biophys Acta 310: 309-16). Hemoglobin was concentrated and sterilized by filtration. Protein solution was dialyzed against ddH₂O and filtered through a 0.45 μm filter. Endotoxins were removed per manufacturer's instructions (Pierce, Rockford, Ill.). Protein solutions were stored at −80° C.

Hemoglobin produced in this method has been previously confirmed to bind oxygen (Shen, T. J., et al. (1993) Proc Natl Acad Sci USA 90: 8108-12). To treat algae with hemoglobin, algae were grown as described and treated to yield a final concentration of 1.5 μM (heme).

B rubrum and C. hydrogenoformans

R. rubrum (American Type Culture Collection, Manassas, Va.) was grown photosynthetically in malate-ammonium medium supplemented with 50 μM NiCl₂ (Kerby, R. L., et al., (1992) J. Bacteriol. 174:5284-94). C. hydrogenoformans (Z-2901/DSM 6008; American Type Culture Collection) were cultivated under strictly anaerobic conditions in a basal carbonate-buffered medium composed as described (Svetlichny V A, et al. (1991) Syst Appl Microbiol 14: 254-260.) Butyl rubber-stoppered bottles of 120 ml contained 50 ml medium.

### Measurement of CODH and Hydrogenase Activity

CODH activity was determined by the reduction of methyl viologen coupled to CO oxidation and the concomitant absorption at 578 nm by UV spectrophotometry. CODH activity was measured in an anaerobic reaction mixture containing 50 mM Tris-HCl, 2 mM methyl viologen and 2 mM dithiothreitol at pH 8.7 (Maness, P. C. and Weaver, P. F. (2002) Int. J. Hydrogen Energy 27:1407-11). The cell free, CODH-enriched fraction was prepared as described (Maness, P. C. and Weaver, P. F. (2001) Appl. Microbiol. Biotechnol. 57:751-6). The supernatant was used as the source of CODH enzyme.

Hydrogenase activity was determined using whole cells by measuring H₂ evolution in an anaerobic reaction mixture containing 2 mM methyl viologen, 0.1% (w/v) Triton X-100 and 50 mM potassium phosphate buffer at pH 7.0. The reaction was initiated by adding 5 mM sodium dithionite. The reaction was incubated 15 minutes at 30° C. in a shaking water bath. The reaction was terminated by addition of 0.1 ml 10% (w/v) tricholoroacetic acid. Hydrogen in the gas phase was determined by gas chromatography as described in Maness, P. C. and Weaver, P. F. (2001) Appl. Microbiol. Biotechnol. 57:751-6.

### Results

Both the addition and expression of hemoglobin increased CODH activity as well as hydrogenase activity. The addition of hemoglobin increased CODH activity from 17.76±2.85 μMol methyl viologen reduction/min/ml to 32.41±4.03 μmol methyl viologen reduction/min/ml (p<9×10⁻⁶). Expression of hemoglobin also increased CODH activity, yielding 29.60±7.30 μMol methyl viologen reduction/min/ml (p<0.005). No significant difference in CODH activity was evident between the addition versus expression of hemoglobin. Similarly, hemoglobin expression increased hydrogenase activity from 137.6±15.0 1 nMol H₂/min/mg cell dry weight to 266.9±27.0 1 nMol H₂/min/mg cell dry weight (p<2×10⁻⁶). Expression of hemoglobin increased hydrogenase activity to 250.4±89.1 nMol H₂/min/mg cell dry weight (p<0.02).

Each of the foregoing patents, patent applications and references that are recited in this application are herein incorporated in their entirety by reference. Having described the presently preferred embodiments, and in accordance with the present invention, it is believed that other modifications, variations and changes will be suggested to those skilled in the art in view of the teachings set forth herein. It is, therefore, to be understood that all such variations, modifications, and changes are believed to fall within the scope of the present invention as defined by the appended claims.

I/we claim:

1. A method for promoting the water-gas shift forward reaction and enhancing hydrogen production in a sample, comprising:
increasing the solubility of CO in the sample,
preventing free radical damage in the sample,
removing or promoting the release of hydrogen or CO₂ from the sample, or adding O₂ to the sample;
so that the water-gas shift forward reaction is promoted and hydrogen production is enhanced.

2. The method of claim 1, wherein the sample is a sample of cells.

3. The method of claim 2, wherein the cells contain CODH.

4.-19. (canceled)

20. A method for promoting the water-gas shift forward reaction and enhancing hydrogen production in a sample of cells, comprising:
increasing the solubility of CO in the sample of cells,
preventing free radical damage in the sample or promoting cell survivability,
regulating the redox potential of the cells in the sample,
removing or promoting the release of hydrogen or CO₂ from the sample of cells, or adding O₂ to the sample of cells;
so that the water-gas shift forward reaction is promoted and hydrogen production is enhanced.

21.-34. (canceled)

35. A method for promoting the water-gas shift forward reaction, comprising:
providing an agent the promotes the water-gas shift forward reaction to a sample containing CODH in an amount effective to promote the water-gas shift forward reaction.

36.-38. (canceled)

39. A method for enhancing hydrogen production, comprising:
promoting the water-gas shift forward reaction in a sample of CODH-containing cells so that hydrogen production is enhanced.

40.-41. (canceled)

42. A method of increasing the solubility of carbon monoxide (CO) in a sample containing CO, comprising:
providing a CO-binding agent to the sample containing CO in an amount effective to increase the solubility of the CO.

43.-55. (canceled)

56. A method of increasing CODH activity, comprising:
increasing the solubility of CO in a sample that contains CODH.

57.-58. (canceled)

59. A method of increasing CODH activity, comprising:
providing a CO-binding agent to a sample that contains CODH, wherein the CODH activity is increased.

60.-67. (canceled)

68. A method of increasing CODH activity, comprising:
removing or promoting the release of hydrogen and/or CO₂ from a sample containing CODH, wherein CODH activity is increased.

69.-74. (canceled)

75. A method of increasing CODH activity, comprising:
providing O₂ to a sample containing CODH, wherein CODH activity is increased.

76.-80. (canceled)

81. A method of increasing CODH activity, comprising:
providing to a sample containing CODH an agent the prevents free radical damage or promotes cell survivability, wherein CODH activity is increased.

82.-83. (canceled)

84. A method of increasing CODH activity, comprising:
regulating the redox potential of CODH-containing cells in a sample, wherein CODH activity is increased.

85.-96. (canceled)

97. A method of enhanced hydrogen production or CO elimination, comprising:
providing a CODH-containing composition in which the water-gas shift forward reaction is promoted to a composition in which enhanced hydrogen production or CO elimination is desired.

98.-107. (canceled)

108. A composition, comprising:
a sample containing CODH, wherein the water-gas shift forward reaction is promoted.

109.-112. (canceled)

113. A composition, comprising:
a sample containing CO and an agent that increases the solubility of CO.

114. (canceled)

115. A composition, comprising:
a sample containing CO and an agent that prevents free radical damage or promotes cell survivability.

116. A CODH-containing cell transfected with a nucleic acid that codes for a CO-binding protein.

117. (canceled)

118. A CODH-containing cell transfected with a nucleic acid that codes for a protein that prevents free radical damage or promotes cell survivability.

119. (canceled)

120. A culture of cells containing CODH and an agent that promotes the water-gas shift forward reaction.

121. (canceled)
